(19) United States
(12) Patent Application Publication
(10) Pub. No.: US 2003/0045850 A1
(43) Pub. Date: Mar. 6, 2003

(54) Unitary distribution layer

(75) Inventors: Peter A. Graef, Puyallup, WA (US); Terry M. Grant, Auburn, WA (US); David G. Marsh, Federal Way, WA (US); Daniel T. Bunker, Karhula (FI); Melissa D. Johnson, Seattle, WA (US)

Correspondence Address: WEYERHAEUSER COMPANY, INTELLECTUAL PROPERTY DEPT., CH 1J27, P.O. BOX 9777, FEDERAL WAY, WA, 98063, US

(73) Assignee: Weyerhaeuser Company

(21) Appl. No.: 10/229,301

(22) Filed: Aug. 27, 2002

Related U.S. Application Data
(63) Continuation of application No. 10/021,668, filed on Dec. 7, 2001.
(60) Provisional application No. 60/251,999, filed on Dec. 7, 2000.
(60) Provisional application No. 60/308,072, filed on Jul. 25, 2001.

(51) Int. Cl. A61F 13/15; A61F 13/20

(52) U.S. Cl. 604/375; 604/378; 604/374; 604/368; 604/367

(57) ABSTRACT

A fibrous layer that includes a refined blend of crosslinked cellulosic fibers and noncrosslinked cellulosic fibers. In one embodiment, the layer includes about 85 percent by weight crosslinked fibers and about 15 percent by weight noncrosslinked fibers. An absorbent construct that includes the fibrous layer and a liquid storage layer. Personal care absorbent products that include the distribution layer.

### CROSS-REFERENCES TO RELATED APPLICATIONS

This application claims the benefit of the priority of the filing dates of U.S. patent application Ser. No. 60/251,999, filed Dec. 7, 2000, and U.S. patent application Ser. No. 60/308,072, filed Jul. 25, 2001. Each application is expressly incorporated herein by reference in its entirety.

### FIELD OF THE INVENTION

The present invention relates to an cellulosic fibrous layer for distributing acquired liquid to a storage layer in liquid communication therewith.

### BACKGROUND OF THE INVENTION

Personal care absorbent products, for example, infant diapers, adult incontinence products, and feminine care products, can include liquid acquisition and/or distribution layers that serve to rapidly acquire and then distribute acquired liquid to a storage core for retention. To achieve rapid acquisition and distribution, these layers often include cellulosic fibers. These layers can include crosslinked cellulosic fibers to impart bulk and resilience to the layer, and wood pulp fibers to increase the wicking of liquid within the layer and to facilitate distribution of the liquid throughout the layer and ultimately to another layer, such as a storage layer, that is in liquid communication with the distribution layer. However, despite advances in these layers, the need exists for a more efficient liquid distribution layer that effectively distributes and transfers acquired liquid to an associated storage layer. The present invention seeks to fulfill these needs and provides further related advantages.

### SUMMARY OF THE INVENTION

In one aspect, the present invention provides a fibrous layer that includes a refined blend of crosslinked cellulosic fibers and noncrosslinked cellulosic fibers. In one embodiment, the layer includes about 85 percent by weight crosslinked fibers and about 15 percent by weight noncrosslinked fibers.

In another aspect of the invention, an absorbent construct is provided that includes a liquid distribution layer and a liquid storage layer. The distribution layer includes a refined blend of crosslinked cellulosic fibers and noncrosslinked cellulosic fibers.

In other aspects, the invention provides personal care absorbent products that include the distribution layer, and methods for making the distribution layer.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram of a representative twin-wire forming device and method for making a representative layer of the invention;

FIG. 2 is a schematic diagram of a representative twin-wire forming device and method for making a representative layer of the invention;

FIG. 3 is a graph of wick time, dry tensile, and cantilever stiffness for a representative layer of the invention;

FIG. 4 is a graph of comparing fluid transfer for three representative layers of the invention to a storage layer as a function of time;

FIG. 5 is a bar graph comparing the fourth gush acquisition time for absorbent constructs: control training pant; control pant and representative layer of the invention; control pant with a storage core; and control pant, representative layer of the invention and storage core;

FIG. 6 is a bar graph comparing the overall liquid capacity before leakage for absorbent constructs: control training pant; control pant and representative layer of the invention; control pant with a storage core; and control pant, representative layer of the invention and storage core;

FIG. 7 illustrates the distibution of liquid in a training pant: control training pant; control pant and representative layer of the invention having a basis weight of about 90 gsm; and control pant and representative layer of the invention having a basis weight of about 180 gsm;

FIG. 8 illustrates the distibution of liquid in a training pant: control training pant; control pant with a storage core; control pant, storage layer, and representative layer of the invention having a basis weight of about 90 gsm; and control pant, storage layer, and representative layer of the invention having a basis weight of about 180 gsm

FIG. 9 is a bar graph comparing the third gush acquisition rate for absorbent constructs: control training pant; control pant and representative layer of the invention; control pant with a storage core; and control pant, representative layer of the invention and storage core;

FIG. 10 is a graph comparing acquisition rate as a function of insult number for absorbent constructs: control training pant; control pant and representative layer of the invention; control pant with a storage core; and control pant, representative layer of the invention and storage core;

FIG. 11 is a bar graph comparing the fourth gush rewet for absorbent constructs: control training pant; control pant and representative layer of the invention; control pant with a storage core; and control pant, representative layer of the invention and storage core;

FIGS. 12A-C illustrate cross-sectional views of portions of representative absorbent constructs that include the distribution layer of the invention; and

FIG. 13A-D illustrate cross-sectional views of portions of representative absorbent articles that include the distribution layer of the invention.

### DETAILED DESCRIPTION OF THE PREFERRED EMBODIMENT

In one aspect, the present invention provides a cellulosic fibrous layer that distributes and transfers liquid acquired by the layer to a storage layer that is in liquid communication therewith. The cellulosic fibrous layer of the invention is a distribution layer that can be incorporated into a personal care absorbent product such as an infant diaper, adult incontinent product, or a feminine care product, among others. In a personal care absorbent product, the distribution layer can be used in combination with one or more other layers. Other layers can include, for example, a storage layer for receiving and storing liquid transferred from the distribution layer, or a storage layer and an acquisition layer.

The distribution layer of the invention includes cellulosic fibers. The cellulosic fibers are suitably wood pulp fibers. In one embodiment, the layer includes a combination of crosslinked cellulosic fibers and noncrosslinked cellulosic fibers.

The distribution layer's crosslinked cellulosic fibers impart bulk and resilience to the layer and provide the layer with a generally open structure for distributing liquid. Suitable crosslinked cellulosic fibers include chemically intrafiber crosslinked cellulosic fibers and are described below. The layer includes crosslinked cellulosic fibers in an amount from about 50 to about 90 percent by weight based on the total weight of fibers in the layer. In one embodiment, the layer includes crosslinked cellulosic fibers in an amount from about 75 to about 90 percent by weight based on the total weight of fibers in the layer. In another embodiment, the layer includes about 85 percent by weight crosslinked cellulosic fibers based on the total weight of fibers in the layer. The layer can include refined crosslinked fibers. The layer can include a refined blend of crosslinked and noncrosslinked fibers.

The distribution layer's noncrosslinked fibers enhance the layer's liquid wicking performance. Suitable noncrosslinked cellulosic fibers include wood pulp fibers capable of liquid wicking and are described below. The layer includes noncrosslinked cellulosic fibers in an amount from about 10 to about 50 percent by weight based on the total weight of fibers in the layer. In one embodiment, the layer includes noncrosslinked cellulosic fibers in an amount from about 10 to about 25 percent by weight based on the total weight of fibers in the layer. In another embodiment, the layer includes about 15 percent by weight noncrosslinked cellulosic fibers based on the total weight of fibers in the layer. The noncrosslinked fibers can include softwood fibers (e.g., southern pine fibers) and hardwood fibers (e.g., Westvaco hardwood fibers or eucalyptus fibers).

In one embodiment, the layer includes southern pine pulp fibers commercially available from Weyerhaeuser Company under the designation NB416. In another embodiment, the layer includes southern pine pulp fibers that have been refined. In a further embodiment, the layer includes eucalyptus pulp fibers. In another embodiment, the layer includes a blend of southern pine and eucalyptus fibers. In still another embodiment, the layer includes a blend of eucalyptus fibers and refined southern pine fibers. In yet a further embodiment, the layer includes a refined blend of southern pine and eucalyptus fibers.

For embodiments that include blends of southern pine and eucalyptus fibers, the ratio of southern pine fibers to eucalyptus fibers can range from about 0.5 to about 1.0 to about 1.0 to about 0.5. In one embodiment, the layer includes about 8 percent by weight eucalyptus fibers, about 7 percent by weight southern pine fibers, and about 85 percent by weight crosslinked fibers based on the total weight of fibers in the layer. In another embodiment, the layer includes about 8 percent by weight eucalyptus fibers, about 7 percent by weight refined southern pine fibers, and about 85 percent by weight crosslinked fibers based on the total weight of fibers in the layer. In another embodiment, the layer includes a refined blend of eucalyptus and southern pine fibers, the layer including about 8 percent by weight eucalyptus fibers, about 7 percent by weight southern pine fibers, and about 85 percent by weight crosslinked fibers based on the total weight of fibers in the layer. In yet another embodiment, the layer includes a refined blend of eucalyptus, southern pine, and crosslinked fibers, the layer including about 8 percent by weight eucalyptus fibers, about 7 percent by weight southern pine fibers, and about 85 percent by weight crosslinked fibers based on the total weight of fibers in the layer.

In one embodiment, the distribution layer includes about 85 percent by weight crosslinked fibers, from about 5 to about 15 percent by weight refined southern pine fibers having a Canadian Standard Freeness of about 500, and from about 0 to about 10 percent by weight southern pine fibers. In one embodiment, the crosslinked fibers, refined southern pine fibers, and southern pine fibers are refined as a blend prior to layer formation.

In another embodiment, the distribution layer includes about 85 percent by weight crosslinked fibers, from about 3 to about 5 percent by weight hardwood fibers, and from about 10 to about 12 percent by weight southern pine fibers. In one embodiment, the crosslinked fibers, hardwood fibers, and southern pine fibers are refined as a blend prior to layer formation.

In one embodiment, the distribution layer has a basis weight in the range from about 20 to about 200 g/m². In another embodiment, the distribution layer has a basis weight in the range from about 50 to about 180 g/m². The distribution layer has a density in the range from about 0.1 to about 0.2 g/cm³.

The characteristics of four representative distribution layers are summarized in Tables 1 and 2 below. In Tables 1 and 2, unsoftened Layer A includes a refined blend of crosslinked fibers (85 percent by weight polyacrylic acid crosslinked fibers) and southern pine fibers (15 percent by weight refined fibers, 500 CSF); unsoftened Layer B includes a refined blend of crosslinked fibers (80 percent by weight polyacrylic acid crosslinked fibers) and southern pine fibers (20 percent by weight refined fibers, 500 CSF); unsoftened Layer C includes a refined blend of crosslinked fibers (85 percent by weight DMeDHEU crosslinked fibers, commercially available from Weyerhaeuser Co. under the designation NHB 416) and southern pine fibers (15 percent by weight refined fibers, 500 CSF); and softened (embossed) Layer D includes a refined blend of crosslinked fibers (85 percent by weight DMeDHEU crosslinked fibers) and southern pine fibers (15 percent by weight refined fibers, 500 CSF). As used herein, the term “unsoftened” refers to a layer that has not been subjected to mechanical treatment, such as, for example, calendering, tenderizing, or embossing. The data presented in Table 1 was acquired using a TRI Autoporosimeter Device.

TABLE 1
Performance Characteristics of Representative Distribution Layers.

| | | MD, CD | Peak | | | | | |
|---|---|---|---|---|---|---|---|---|
| | Ring | Gurley | Geometric | MDPsigbloc | | | | Surface |
| | Crush | Stiffness | Mean Tensile | k:MAP* | | | | Tension |
| Layer | (g) | SGU/mm | (g/cm) | Ratio | MDP* | MAP* | MUP* | (dynes/cm) |
| A | 3405 | 1137, 562 | 858.0 | 1.81:1 | 24.2 | 13.4 | 10.0 | 65.5 |
| B | 1500 | 650, 266 | 763.5 | 1.72:1 | 22.1 | 12.9 | 9.5 | 69.6 |
| C | 1500 | 623, 390 | 725.5 | 1.91:1 | 29.0 | 15.2 | 9.2 | 66.8 |
| D | 900 | 351, 163 | 546.5 | 1.98:1 | 28.5 | 14.4 | 8.1 | 66.8 |

TABLE 2
Performance Characteristics of Representative Distribution Layers.

| | | | | | Wicking | | |
|---|---|---|---|---|---|---|---|
| | Ave. O.D. | Ave. A.D. | Wicking | | Capacity at | | |
| | Basis | Basis | Time to | Wicking | 15 cm after | MD, CD | MD, CD |
| | Weight | Weight | 15 cm | Height at 15 | 15 min | Tensile | Elongation |
| Layer | (gsm) | (gsm) | (sec) | min (cm) | (g/g) | (g/cm) | (%) |
| A | 88 | 0.114 | 174 | 21.8 | 8.6 | 1020, 696 | 2.6, 5.6 |
| B | 52 | 0.117 | 291 | 19.8 | 7.3 | 952, 575 | 2.4, 4.1 |
| C | 53 | 0.176 | 277 | 19.2 | 7.7 | 899, 552 | 2.7, 3.8 |
| D | 53 | 0.165 | 326 | 18.6 | 7.5 | 651, 442 | 2.8, 5.1 |

In addition to cellulosic fibers, the distribution layer can include a wet strength agent. Suitable wet strength agents are described below. The wet strength agent is present in the layer in an amount from about 5 to about 20 pounds/ton fiber. In one embodiment, the wet strength agent is a polyamide-epichlorohydrin resin present in the layer in about 10 pounds/ton fiber.

As noted above, the distribution layer of the invention includes crosslinked cellulosic fibers. Any one of a number of crosslinking agents and crosslinking catalysts, if necessary, can be used to provide the crosslinked fibers to be included in the layer. The following is a representative list of useful crosslinking agents and catalysts. Each of the patents noted below is expressly incorporated herein by reference in its entirety.

Suitable urea-based crosslinking agents include substituted ureas such as methylolated ureas, methylolated cyclic ureas, methylolated lower alkyl cyclic ureas, methylolated dihydroxy cyclic ureas, dihydroxy cyclic ureas, and lower alkyl substituted cyclic ureas. Specific urea-based crosslinking agents include dimethyldihydroxy urea (DMDHU, 1,3-dimethyl-4,5-dihydroxy-2-imidazolidinone), dimethyloldihydroxyethylene urea (DMDHEU, 1,3-dihydroxymethyl-4,5-dihydroxy-2-imidazolidinone), dimethylol urea (DMU, bis[N-hydroxymethyl]urea), dihydroxyethylene urea (DHEU, 4,5-dihydroxy-2-imidazolidinone), dimethylolethylene urea (DMEU, 1,3-dihydroxymethyl-2-imidazolidinone), and dimethyldihydroxyethylene urea (DMeDHEU or DDI, 4,5-dihydroxy-1,3-dimethyl-2-imidazolidinone).

Suitable crosslinking agents include dialdehydes such as C₂-C₈ dialdehydes (e.g., glyoxal), C₂-C₈ dialdehyde acid analogs having at least one aldehyde group, and oligomers of these aldehyde and dialdehyde acid analogs, as described in U.S. Pat. Nos. 4,822,453; 4,888,093; 4,889,595; 4,889,596; 4,889,597; and 4,898,642. Other suitable dialdehyde crosslinking agents include those described in U.S. Pat. Nos. 4,853,086; 4,900,324; and 5,843,061.

Other suitable crosslinking agents include aldehyde and urea-based formaldehyde addition products. See, for example, U.S. Pat. Nos. 3,224,926; 3,241,533; 3,932,209; 4,035,147; 3,756,913; 4,689,118; 4,822,453; 3,440,135; 4,935,022; 3,819,470; and 3,658,613.

Suitable crosslinking agents include glyoxal adducts of ureas, for example, U.S. Pat. No. 4,968,774, and glyoxal/cyclic urea adducts as described in U.S. Pat. Nos. 4,285,690; 4,332,586; 4,396,391; 4,455,416; and 4,505,712.

Other suitable crosslinking agents include carboxylic acid crosslinking agents such as polycarboxylic acids. Polycarboxylic acid crosslinking agents (e.g., citric acid, propane tricarboxylic acid, and butane tetracarboxylic acid) and catalysts are described in U.S. Pat. Nos. 3,526,048; 4,820,307; 4,936,865; 4,975,209; and 5,221,285. The use of C₂-C₉ polycarboxylic acids that contain at least three carboxyl groups (e.g., citric acid and oxydisuccinic acid) as crosslinking agents is described in U.S. Pat. Nos. 5,137,537; 5,183,707; 5,190,563; 5,562,740, and 5,873,979.

Polymeric polycarboxylic acids are also suitable crosslinking agents. Suitable polymeric polycarboxylic acid crosslinking agents are described in U.S. Pat. Nos. 4,391,878; 4,420,368; 4,431,481; 5,049,235; 5,160,789; 5,442,899; 5,698,074; 5,496,476; 5,496,477; 5,728,771; 5,705,475; and 5,981,739. Polyacrylic acid and related copolymers as crosslinking agents are described U.S. Pat. Nos. 5,549,791 and 5,998,511. Polymaleic acid crosslinking agents are described in U.S. Pat. No. 5,998,511.

Specific suitable polycarboxylic acid crosslinking agents include citric acid, tartaric acid, malic acid, succinic acid, glutaric acid, citraconic acid, itaconic acid, tartrate monosuccinic acid, maleic acid, polyacrylic acid, polymethacrylic acid, polymaleic acid, polymethylvinylether-co-maleate copolymer, polymethylvinylether-co-itaconate copolymer, copolymers of acrylic acid, and copolymers of maleic acid.

Other suitable crosslinking agents are described in U.S. Pat. Nos. 5,225,047; 5,366,591; 5,556,976; and 5,536,369.

Suitable catalysts can include acidic salts, such as ammonium chloride, ammonium sulfate, aluminum chloride, magnesium chloride, magnesium nitrate, and alkali metal salts of phosphorous-containing acids. In one embodiment, the crosslinking catalyst is sodium hypophosphite.

Mixtures or blends of crosslinking agents and catalysts can also be used.

The crosslinking agent is applied to the cellulosic fibers in an amount sufficient to effect intrafiber crosslinking. The amount applied to the cellulosic fibers can be from about 1 to about 10 percent by weight based on the total weight of fibers. In one embodiment, crosslinking agent in an amount from about 4 to about 6 percent by weight based on the total weight of fibers.

In addition to crosslinked fibers, the distribution layer of the invention also includes noncrosslinked cellulosic fibers. Suitable cellulosic fibers include those known to those skilled in the art and include any fiber or fibrous mixture from which a fibrous web or sheet can be formed.

Although available from other sources, cellulosic fibers are derived primarily from wood pulp. Suitable wood pulp fibers for use with the invention can be obtained from well-known chemical processes such as the kraft and sulfite processes, with or without subsequent bleaching. Pulp fibers can also be processed by thermomechanical, chemithermomechanical methods, or combinations thereof. The preferred pulp fiber is produced by chemical methods. Groundwood fibers, recycled or secondary wood pulp fibers, and bleached and unbleached wood pulp fibers can be used. Softwoods and hardwoods can be used. Details of the selection of wood pulp fibers are well known to those skilled in the art. These fibers are commercially available from a number of companies, including Weyerhaeuser Company, the assignee of the present invention. For example, suitable cellulose fibers produced from southern pine that are usable with the present invention are available from Weyerhaeuser Company under the designations CF416, NF405, PL416, FR516, and NB416.

The wood pulp fibers useful in the present invention can also be pretreated prior to use. This pretreatment may include physical treatment, such as subjecting the fibers to steam, or chemical treatment. Other pretreatments include incorporation of antimicrobials, pigments, dyes and densification or softening agents. Fibers pretreated with other chemicals, such as thermoplastic and thermosetting resins also may be used. Combinations of pretreatments also may be employed. Treatments can also be applied after formation of the fibrous product in post-treatment processes, examples of which include the application of surfactants or other liquids, which modify the surface chemistry of the fibers, and the incorporation of antimicrobials, pigments, dyes, and densification or softening agents.

The distribution layer optionally includes a wet strength agent. Suitable wet strength agents include cationic modified starch having nitrogen-containing groups (e.g., amino groups) such as those available from National Starch and Chemical Corp., Bridgewater, N.J.; latex; wet strength resins, such as polyamide-epichlorohydrin resin (e.g., KYMENE 557LX, Hercules, Inc., Wilmington, Del.), and polyacrylamide resin (see, e.g., U.S. Pat. No. 3,556,932 and also the commercially available polyacrylamide marketed by American Cyanamid Co., Stanford, Conn., under the trade name PAREZ 631 N.C.); urea formaldehyde and melamine formaldehyde resins; and polyethylenimine resins. A general discussion on wet strength resins utilized in the paper field, and generally applicable in the present invention, can be found in TAPPI monograph series No. 29, “Wet Strength in Paper and Paperboard”, Technical Association of the Pulp and Paper Industry (New York, 1965).

In another aspect of the invention, methods for forming the distribution layer are provided. Representative distribution layers can be formed using conventional papermaking machines including, for example, Rotoformer, Fourdrinier, inclined wire Delta former, and twin-wire machines.

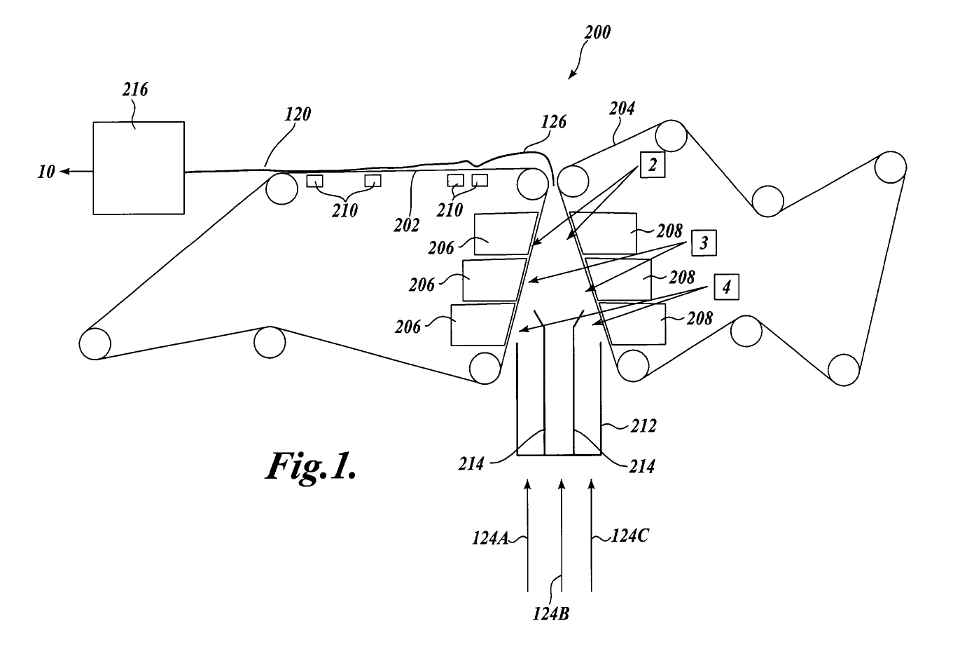

The layer can be formed by devices and processes that include a twin-wire configuration (i.e., twin-forming wires). Representative forming methods applicable for forming the distribution layer of the invention are described in PCT/US99/05997 (Method for Forming a Fluted Composite) and PCT/US99/27625 (Reticulated Absorbent Composite), each incorporated herein by reference in its entirety. A representative twin-wire machine for forming the layer is shown in FIG. 1. Referring to FIG. 1, machine 200 includes twin-forming wires 202 and 204 onto which the layer's components are deposited. Basically, fibrous slurry 124 is introduced into headbox 212 and deposited onto forming wires 202 and 204 at the headbox exit. Vacuum elements 206 and 208 dewater the fibrous slurries deposited on wires 202 and 204, respectively, to provide partially dewatered webs that exit the twin-wire portion of the machine as partially dewatered web 126. Web 126 continues to travel along wire 202 and continues to be dewatered by additional vacuum elements 210 to provide wet composite 120 which is then dried by drying means 216 to provide layer 10.

In one embodiment, the composite is formed by a wetlaid process using the components described above. The wetlaid method can be practiced on an inclined wire Delta former. In another embodiment, the composite is formed by a foam-forming method using the components described above. Wetlaid and foam-forming processes can be practiced on a twin-wire former.

A representative method for forming a distribution layer of the invention includes the following steps:

(a) forming a fibrous slurry comprising fibers in an aqueous dispersion medium; for a foam method, the slurry is a foam that includes, in addition to fibers, a surfactant;

(b) moving a first foraminous element (e.g., a forming wire) in a first path;

(c) moving a second foraminous element in a second path;

(d) passing a first portion of the slurry into contact with the first foraminous element moving in a first path;

(e) passing a second portion of the slurry into contact with the second foraminous element moving in the second path; and

(f) forming a fibrous web from the slurry by withdrawing liquid from the slurry through the first and second foraminous elements.

As noted above, the foam-forming method is suitably carried out on a twin-wire former, preferably a vertical former, and more preferably, a vertical downflow twin-wire former. In the vertical former, the paths for the foraminous elements are substantially vertical.

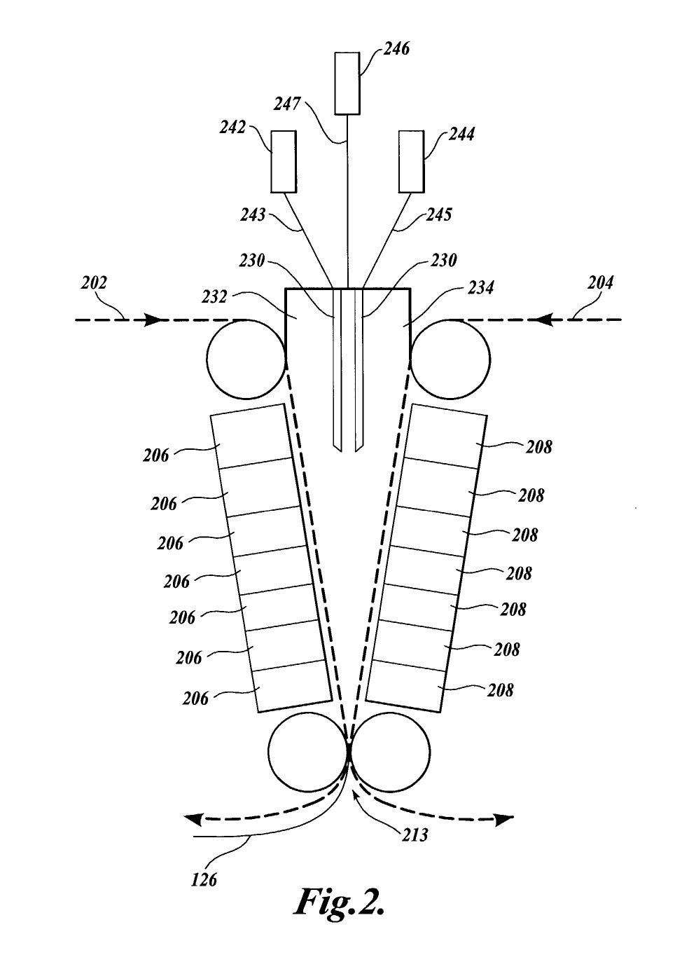

A representative vertical downflow twin-wire former useful in practicing a method of the invention is illustrated in FIG. 2. Referring to FIG. 2, the former includes a vertical headbox assembly having a former with a closed first end (top), closed first and second sides and an interior volume. A second end (bottom) of the former is defined by moving first and second foraminous elements, 202 and 204, and forming nip 213. The interior volume defined by the former's closed first end, closed first and second sides, and first and second foraminous elements includes an interior structure 230 extending from the former first end and toward the second end. The interior structure defines a first volume 232 on one side thereof and a second volume 234 on the other side thereof. The former further includes supply 242 and means 243 for introducing a first fiber/foam slurry into the first volume, supply 244 and means 245 for introducing a second fiber/foam slurry into the second volume, and supply 246 and means 247 for introducing a third material (e.g., the first or second fiber/foam slurry) into the interior structure. Means for withdrawing liquid/foam (e.g., suction boxes 206 and 208) from the first and second slurries through the foraminous elements to form a web are also included in the headbox assembly.

In the method, the twin-wire former includes a means for introducing at least a third material (e.g., the first or second fiber/foam slurry) through the interior structure. The first and second fiber/foam slurries can include the same components (e.g., crosslinked cellulosic fibers, southern pine fibers, eucalyptus fibers) and have the same composition.

Depending upon the nature of the composite to be formed, the first and second fiber/foam slurries may be the same as or different from each other, and the same as or different from a third material.

The means for withdrawing liquid/foam from the first and second slurries through the foraminous elements to form a web on the foraminous elements are also included in the headbox assembly. The means for withdrawing liquid/foam can include any conventional means for that purpose, such as suction rollers, pressing rollers, or other conventional structures. In a preferred embodiment, first and second suction box assemblies are provided and mounted on the opposite sides of the interior structure from the foraminous elements (see boxes 206 and 208 in FIGS. 1 and 2).

The distribution layer of the invention advantageously exhibits strength (e.g., structural integrity) and softness. In addition to having flexibility and softness suitable for incorporation into personal care absorbent products, the composites of the invention exhibit advantageous structural integrity. Structural integrity can be indicated by tensile strength. Suitable layers have a tensile strength greater than about 10 N/50 mm.

Suitable layers have a machine direction (MD) tear strength greater than about 205 mN, and a cross-machine direction (CD) tear strength greater than about 260 mN. The tear strength of representative distribution layers of the invention was determined by ASTM Method No. P-326-5. In the method, the machine direction (MD) and cross-machine direction (CD) tear strengths of 10 specimens of representative layers (1-3 in Table 1 below) were measured. Layer 1 included 85 percent by weight crosslinked fibers, 8 percent by weight eucalyptus fibers, and 7 percent by weight southern pine fibers. Layer 2 included 85 percent by weight crosslinked fibers, 8 percent by weight eucalyptus fibers, and 7 percent by weight refined southern pine fibers. Layer 3 included 85 percent by weight crosslinked fibers, 8 percent by weight hardwood fibers (Westvaco), and 7 percent by weight refined southern pine fibers. The average, maximum, minimum tear strengths as well as their ranges (mN) are summarized in Table 3.

TABLE 3
Representative Distribution Layer Tear Strength.

| Layer | Average | Maximum | Minimum | Range |
|---|---|---|---|---|
| 1 (MD) | 242.2 | 284.4 | 215.7 | 68.6 |
| 1 (CD) | 322.6 | 362.8 | 304.0 | 58.8 |
| 2 (MD) | 419.7 | 431.5 | 402.1 | 29.4 |
| 2 (CD) | 531.5 | 559.0 | 490.3 | 68.6 |
| 3 (MD) | 388.3 | 431.5 | 362.8 | 68.6 |
| 3 (CD) | 514.8 | 588.4 | 460.9 | 127.5 |

Extracts of suitable layers have a surface tension greater than about 50 dynes/cm. The method for determining the surface tension of a pulp extract is described below.

Suitable layers have a softness, as measured by ring crush, less than about 1200 g.

The distribution layer of the invention exhibits advantageous fluidic properties. The properties can be indicated by various measures including liquid acquisition rate, rewet, wicking, mid-point desorption pressure, mid-point acquisition pressure, and mid-point uptake.

The layer has a mid-point desorption pressure (MDP) greater than about 20 cm. In one embodiment, the layer has a MDP greater than about 30 cm. In another embodiment, the layer has a MDP greater than about 40 cm.

The layer has a mid-point acquisition pressure (MAP) less than about 25 cm. In one embodiment, the layer has a MAP less than about 20 cm.

The layer has a mid-point uptake (MU) greater than about 5 g/g.

A description of the method for determining MDP, MAP, and MU is provided in Liquid Porosimetry: New Methodology and Applications, B. Miller and I. Tomkin, Journal of Colloid Interface Science, 162:163-170, 1994, incorporated herein by reference in its entirety.

Liquid transfer rate was determined by soaking a strip of representative distribution layer (10 cm width) with synthetic urine. The soaked layer was allowed to drain for 3 minutes on the test device. The test device on which the layer was placed included a horizontal surface adjacent a 60 degree sloped surface (i.e., a ramp). The distribution layer extended across the horizontal and sloped portions of the device with one end terminating in a reservoir containing a known amount of synthetic urine. The horizontal surface was 11 cm above the lower edge of the sloped surface. A receiving layer (e.g., storage layer, 10 cm×10 cm) was placed on top of the distribution layer on the horizontal surface. A weight (704 g, 10 cm×10 cm delivering 0.10 psi) was placed on top of the receiving layer. The receiving layer was allowed to absorb for 20 minutes against the 15 cm head. The amount of liquid transferred from the reservoir was measured and the transfer rate calculated.

The layer of the invention provides a liquid transfer rate greater than zero at a wicking height of 11 cm when incorporated as the distribution layer into a commercial infant diaper (PAMPERS).

Other physical and performance characteristics of representative distribution layers of the invention (Layers 4-8) are summarized in Table 4 below. Layer 4 included 85 percent by weight crosslinked fibers, 8 percent by weight eucalyptus fibers, and 7 percent by weight southern pine fibers. Layers 5-8 were derived from Layer 4 by softening under varying conditions (4, 12, 16, and 17, respectively) as described below in Table 4. Layer 5 was softened by applying a pressure of 35 bar with a cold calender roll; Layer 6 was softened by applying a pressure of 35 bar with a cold calender roll and 2 bar in the layer's machine direction; Layer 7 was softened by applying a pressure of 35 bar with a cold calender roll and embossing the top and bottom surfaces of the layer (2 passes) at a pressure of 8 bar; and Layer 8 was softened by applying a pressure of 8 bar to the layer's machine and cross-machine directions.

TABLE 4
Representative Distribution Layer Physical and Performance Characteristics.

| | Distribution Layer | | | | |
|---|---|---|---|---|---|
| Test | 4 | 5 | 6 | 7 | 8 |
| Capsorption | | | | | |
| MDP (cm) | 32.2 | 44.2 | 43.5 | 42 | 35.3 |
| MAP (cm) | 17.5 | 23.6 | 22.3 | 22.3 | 18.8 |
| MU (g/g) | 7 | 5.4 | 5.8 | 5.3 | 6.8 |
| Softness (ring crush, g) | 2700 | 1070 | 320 | 330 | 250 |
| Tensile (N/50 mm) | 29.2 | 20.8 | 12.2 | 8.9 | 2.3 |
| Surface tension | 48 | 53 | 52 | 52 | 53 |
| Brightness | 72.2 | 73.7 | 73.7 | 74.1 | 73.1 |
| Basis weight (g/m²) | 152 | 152 | 153 | 153 | 137 |
| Caliper (mm) | 1.29 | 0.54 | 0.77 | 0.72 | 1.30 |
| Density (g/cm³) | 0.118 | 0.283 | 0.200 | 0.212 | 0.105 |
| Wicking time to 15 cm (sec) | 273 | 238 | 240 | 248 | 710 |
| Wick capacity @ 15 cm (g/g) | 6.6 | 6 | 6.2 | 6.4 | 7.1 |
| Wicked Ht. @ 15 min (cm) | 19.2 | 21 | 21.2 | 20.2 | 15.2 |
| Softness | | | | | |
| Cantilever Stiffness, MD (mm) | 107 | 59 | 53 | 41 | 39 |
| Cantilever Stiffness, CD (mm) | 83 | 51 | 29 | 27 | 37 |
| Strength | | | | | |
| Dry Tensile, MD (N/50 mm) | 292 | 20.8 | 12.2 | 8.9 | 2.3 |
| Dry Elong. (mm) | 4.3 | 4.9 | 5.5 | 6.5 | 9.7 |
| Dry Elong. (%) | 2.1 | 2.5 | 2.7 | 3.2 | 4.8 |
| Wet Tensile, MD (N/50 mm) | 8.9 | 5.1 | 3.4 | 2.1 | 0.7 |
| Wet Elong. (mm) | 11.3 | 12.4 | 13.3 | 13.1 | 10.4 |
| Wet Elong. (%) | 5.7 | 6.2 | 6.7 | 6.6 | 5.2 |
| Wet Strength (W/D %) | 31 | 25 | 28 | 24 | 28 |
| Capacity (g/g pad) | 3.8 | 3.6 | 3.6 | 3.8 | 3.7 |

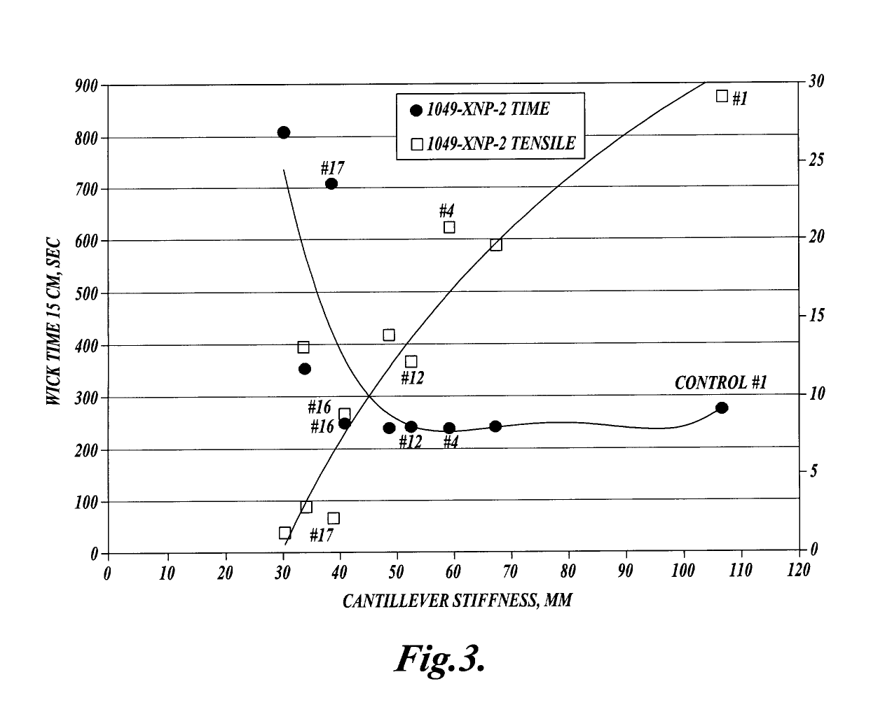

Wick time and tensile versus cantilever stiffness for Layers 4-8 is illustrated graphically in FIG. 3.

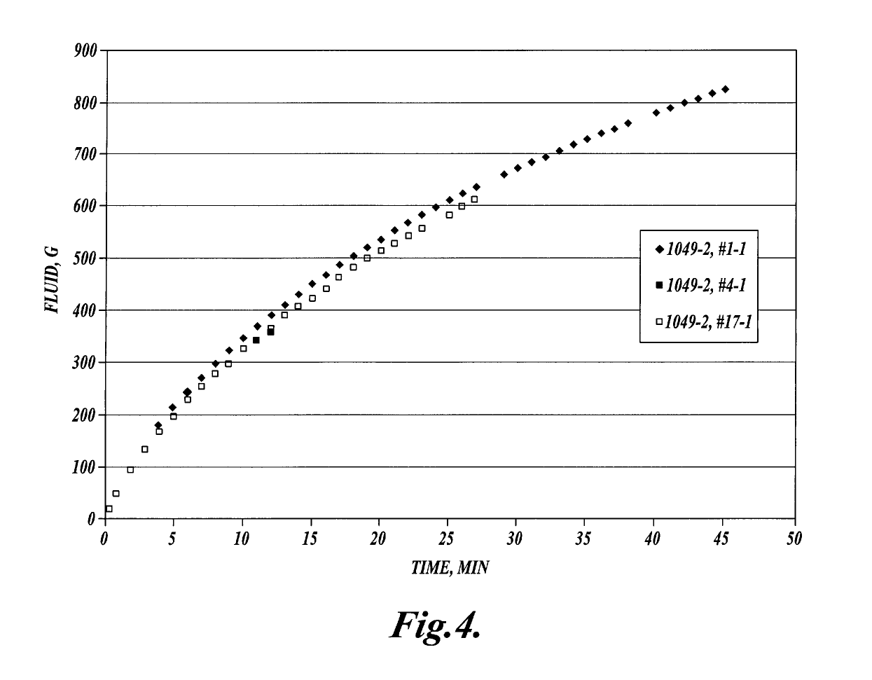

Fluid transfer to core versus time for Layers 4, 5, and 8 is illustrated graphically in FIG. 4.

The distribution layer formed in accordance with the present invention can be incorporated into an absorbent article such as a diaper. The composite can be used alone or combined with one or more other layers, such as acquisition and/or storage layers, to provide useful absorbent constructs.

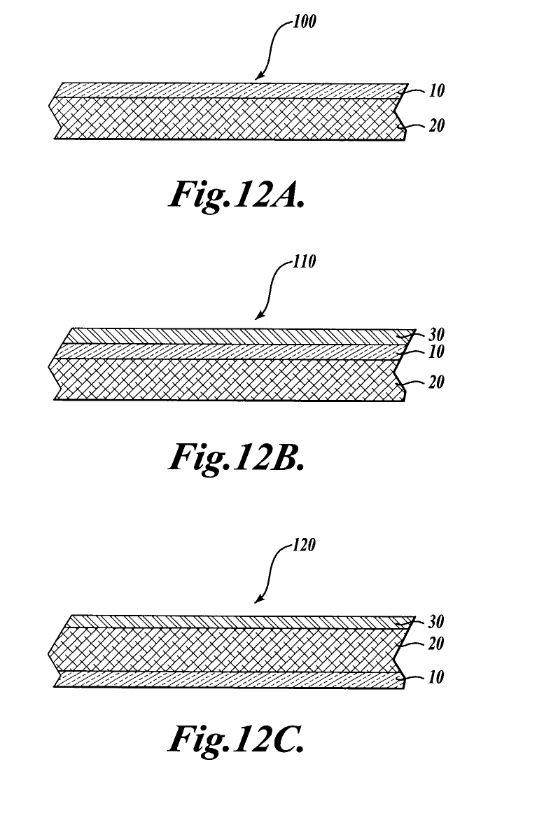

Representative absorbent constructs that incorporate the distribution layer are illustrated in FIGS. 12A-C. Referring to FIG. 12A, representative distribution layer 10 can be combined with a storage layer 20 to provide construct 100. Referring to FIG. 12B, acquisition layer 30 can be combined with distribution layer 10 and storage layer 20 to provide construct 110 having distribution layer 10 intermediate acquisition layer 30 and storage layer 20. Referring to FIG. 12C, acquisition layer 30 can be combined with distribution layer 10 and storage layer 20 to provide construct 120 having storage layer 20 intermediate acquisition layer 30 and distribution layer 10.

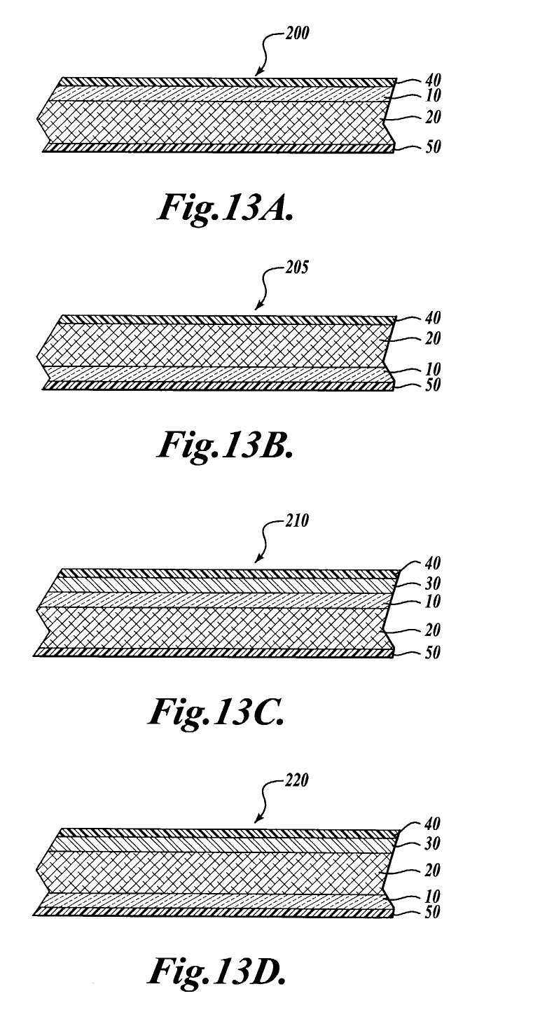

As noted above, the distribution layer can be incorporated into personal care absorbent products, such as infant diapers, training pants, and incontinence products. Representative absorbent articles that incorporate the distribution layer are illustrated in FIGS. 13A-D. In general, the absorbent articles include an absorbent construct intermediate a liquid pervious face sheet and a liquid impervious back sheet. Typically, in such absorbent articles, the face sheet is joined to the back sheet. Referring to FIG. 13A, article 200 includes face sheet 40, distribution layer 10, storage layer 20, and back sheet 50. In this article, distribution layer 10 is adjacent face sheet 40. Referring to FIG. 13B, article 205 includes face sheet 40, storage layer 20, distribution layer 10, and back sheet 50 with distribution layer 10 adjacent back sheet 50. Referring to FIG. 13C, article 210 includes face sheet 40, acquisition layer 30, distribution layer 10, storage layer 20, and back sheet 50. In this article, distribution layer 10 is intermediate acquisition layer 30 and storage layer 20. Referring to FIG. 13D, article 220 includes face sheet 40, acquisition layer 30, storage layer 20, distribution layer 10, and back sheet 50. In this article, distribution layer 10 is adjacent back sheet 50.

It will be appreciated that absorbent constructs and articles that include the distribution layer of the invention can have a variety of designs and are within the scope of this invention.

The distribution layer was tested in training pants.

In the following tests the training pants contain SAP. As used herein, a SAP or “superabsorbent particles” or “superabsorbent material” refers to a polymeric material that is capable of absorbing large quantities of fluid by swelling and forming a hydrated gel (i.e., a hydrogel). In addition to absorbing large quantities of fluids, superabsorbent materials can also retain significant amounts of bodily fluids under moderate pressure.

Superabsorbent materials generally fall into three classes: starch graft copolymers, crosslinked carboxymethylcellulose derivatives, and modified hydrophilic polyacrylates. Examples of such absorbent polymers include hydrolyzed starch-acrylonitrile graft copolymers, neutralized starch-acrylic acid graft copolymers, saponified acrylic acid ester-vinyl acetate copolymers, hydrolyzed acrylonitrile copolymers or acrylamide copolymers, modified crosslinked polyvinyl alcohol, neutralized self-crosslinking polyacrylic acids, crosslinked polyacrylate salts, carboxylated cellulose, and neutralized crosslinked isobutylene-maleic anhydride copolymers.

Superabsorbent materials are available commercially, for example, polyacrylates from Clariant of Portsmouth, Va. These superabsorbent polymers come in a variety of sizes, morphologies, and absorbent properties (available from Clariant under trade designations such as IM 3500 and IM 3900). Other superabsorbent materials are marketed under the trademarks SANWET (supplied by Sanyo Kasei Kogyo Kabushiki Kaisha), and SXM77 (supplied by Stockhausen of Greensboro, N.C.). Other superabsorbent materials are described in U.S. Pat. No. 4,160,059; U.S. Pat. No. 4,676,784; U.S. Pat. No. 4,673,402; U.S. Pat. No. 5,002,814; U.S. Pat. No. 5,057,166; U.S. Pat. No. 4,102,340; and U.S. Pat. No. 4,818,598, all expressly incorporated herein by reference. Products such as diapers that incorporate superabsorbent materials are described in U.S. Pat. No. 3,699,103 and U.S. Pat. No. 3,670,731.

The first control training pant was a large “Members Mark” Kids Pants (Paragon Training Pant) which has a storage core containing approximately 46% SAP. The storage core has a capacity of approximately 380 mls (milliliters) of urine. The core contains 13 grams of SAP mixed with 15 grams of airlaid fluff pulp.

This control was compared to two test training pants. Each of the test training pants used the same control training pant. In each of the test training pants a distribution layer was placed under the storage core.

In the first test training pant, also called Paragon Training Pant with UDL 1049-5, the UDL distribution layer had a weight of 180 gsm (grams per square meter) and a capacity of 48 mls of urine. It contained 8 grams of fiber.

In the second test pant, also called Paragon Training Pant with UDL 1081-8, the UDL distribution layer had a weight of 90 gsm and a capacity of 24 mls of urine. It contained 4 grams of fiber.

The second control training pant was a large “Members Mark” Kids Pants (Paragon Training Pant with 70% core) which has a storage core containing approximately 70% SAP. The storage core has a capacity of approximately 320 mls of urine. The core contains 13 grams of SAP mixed with 5.5 grams of airlaid treated fluff pulp. The pulp was mixed with a mixture of equal molecular amounts of propylene glycol, lactic acid and sodium lactate. The amount of the mixture on the pulp was 7-9% of the weight of the pulp.

This control was also compared to two test training pants. Each of the test training pants used the same control training pant. In each of the test training pants a distribution layer was placed tinder the storage core.

In the first test training pant, also called Paragon Training Pant with 70% core and UDL 1049-5, the UDL distribution layer had a weight of 180 gsm and a capacity of 48 mls of urine. It contained 8 grams of fiber.

In the second test pant, also called Paragon Training Pant with 70% core and UDL 1081-8, the UDL distribution layer had a weight of 90 gsm and a capacity of 24 mls of urine. It contained 4 grams of fiber.

### Saddle Wicking Test

Saddle wicking, including acquisition rate, distribution, and wicking height, was determined by the method described below.

Procedure:

1) Draw and label the 6 even cells using a template and a permanent marker.

2) Place an “X” at the midpoint of the line between the 3^{rd} and 4^{th} cells.

3) Position diaper in Saddle Device so that the “X” is squarely at the bottom of the apparatus and then position a 250 ml separatory funnel approximately 1 cm directly above the “X.”

4) Measure out 75 ml of synthetic urine (Blood Bank 0.9% saline) and pour into funnel.

5) Open the funnel and start the timer. Measure the time at which all of the fluid has left the funnel to the point where the fluid is absorbed into the sample. Record as acquisition time.

6) Repeat steps 7 and 8 every 20 minutes, until the training pant leaks (Free fluid in training pant 20 minutes after the insult or fluid addition)

7) When the diaper has leaked extract the free fluid out of the training pant using a syringe.

8) Measure and record the amount of free fluid extracted in step 7.

9) Pull out training pant and cut sample into designated cells.

10) Weigh each cell and record the wet weight.

11) Place each cell into oven to dry.

12) Weigh and record dry weights of each cell.

13) Calculate the amount of fluid in each cell (wet weight—dry weight).

14) Calculate the capacity utilized before leakage ((number of insults×75 ml)—free fluid extracted).

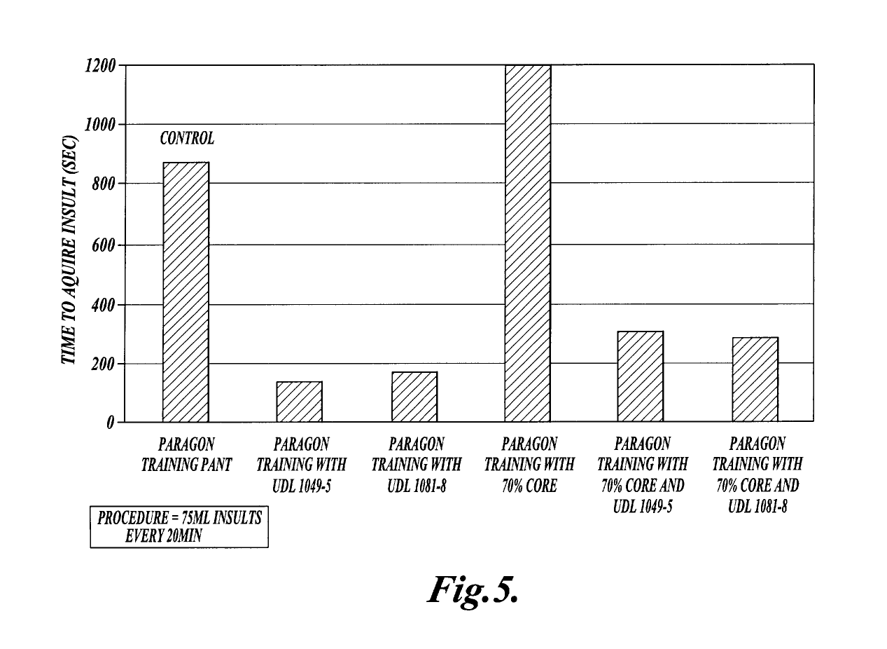

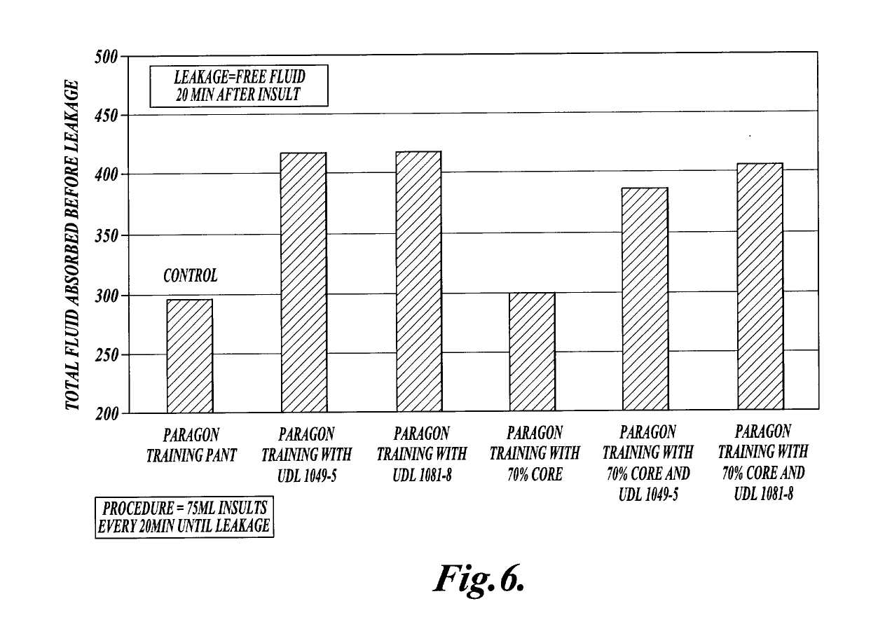

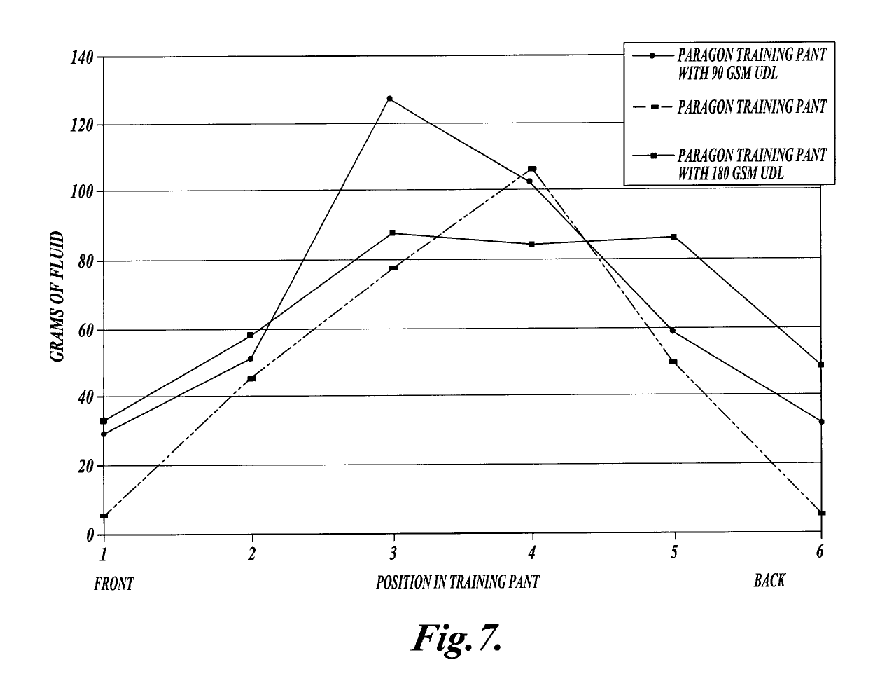

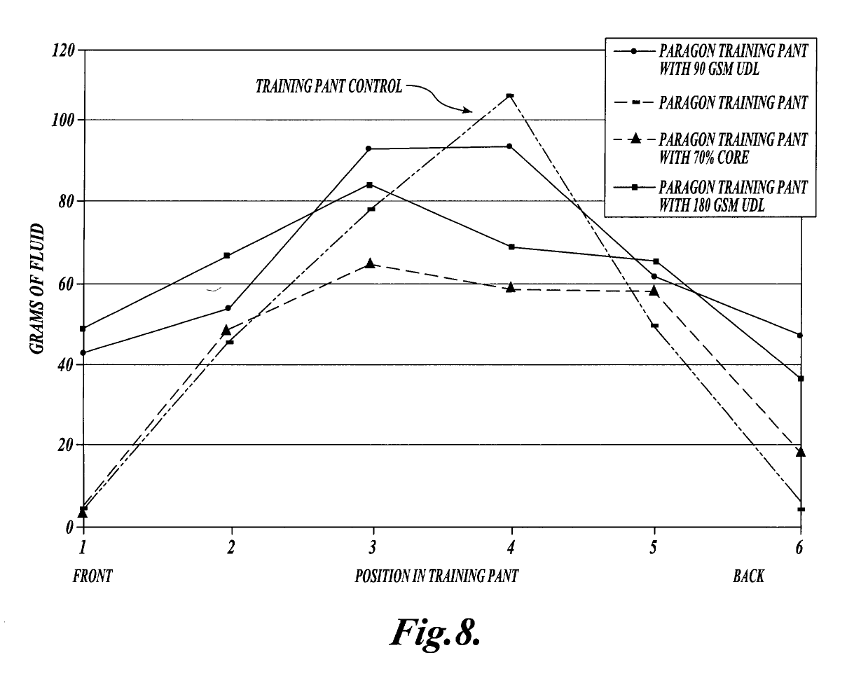

The results of the saddle wicking tests are shown in FIGS. 5 through 11. FIG. 5 shows the time in seconds to acquire fluid during the 4^{th} insult for the control and test training pants, and demonstrates the effectiveness of the UDL in transferring fluid so the core can acquire fluid more rapidly. FIG. 6 shows the total fluid absorbed in milliliters before leakage occurred. FIGS. 7 and 8 show the distribution of fluid in grams in each of the zones of the training pant.

### Market Pulp Flat Acquisition Test

Acquisition time and rewet were obtained for the control and test training pants.

The acquisition time and rewet are determined in accordance with the multiple-dose rewet test described below.

Briefly, the multiple-dose rewet test measures the amount of synthetic urine released from. an absorbent structure after each of three liquid applications, and the time required for each of the three liquid doses to wick into the product.

The aqueous solution used in the tests was a synthetic urine made up of one part synthetic urine concentrate and nine parts deionized water.

The training pant was clamped onto a clampboard, fully extended, with the nonwoven side up. The training pant was prepared for the test by determining the center of the structure's core, measuring 2.5 cm. to the front for liquid application location, and marking the location with an “X”. A dosing ring (5/32 inch stainless steel, 2 inch ID×3 inch height) was placed onto the “X” marked on the samples. A liquid application funnel (minimum 100 mL capacity, 5-7 mL/s flow rate) was placed 2-3 cm. above the dosing ring at the “X”. Once the sample was prepared, the test was conducted as follows.

The funnel was filled with a dose (75 mL) of synthetic urine. A first dose of synthetic urine was applied within the dosing ring. Using a stopwatch, the liquid acquisition time was recorded in seconds from the time the funnel valve was opened until the liquid wicked into the product from the bottom of the dosing ring. The acquisition rate was determined by dividing the amount of synthetic urine (75 ml) by the acquisition time to obtain the acquisition rate in grams per second. A milliliter of synthetic urine is equal to 1 gram.

After a twenty-minute wait period, rewet was determined. During the twenty-minute wait period after the first dose was applied, a stack of filter papers (19-22 g, Whatman #3, 11.0 cm or equivalent, that had been exposed to room humidity for minimum of 2 hours before testing) was weighed. The stack of preweighed filter papers was placed on the center of the wetted area. A cylindrical weight (8.9 cm diameter, 9.8 lb.) was placed on top of these filter papers. After two minutes the weight was removed, the filter papers were weighed and the weight change recorded.

The procedure was repeated two more times. Another 75 ml dose of synthetic urine was added to the diaper, and the acquisition time and rate was determined, filter papers were placed on the sample for two minutes, and the weight change determined. For the second dose, the weight of the dry filter papers was 29-32 g, and for the third dose, the weight of the filter papers was 39-42 g. The dry papers from the prior dosage were supplemented with additional dry filter papers.

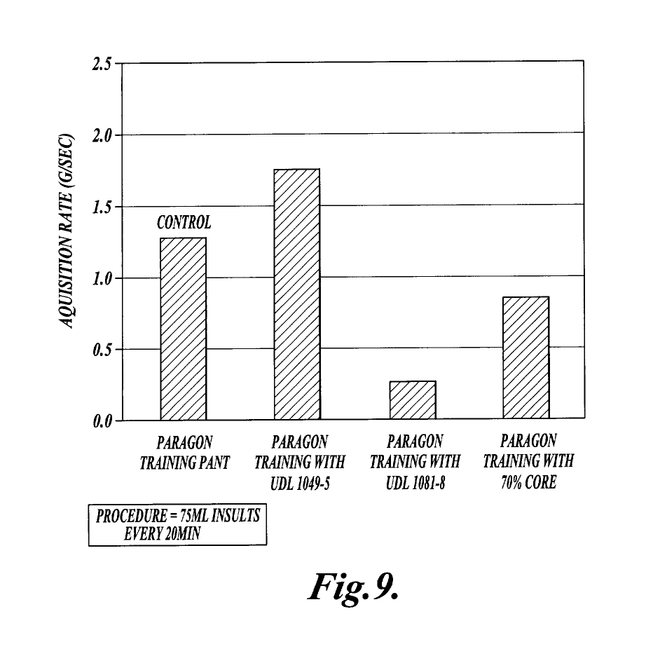

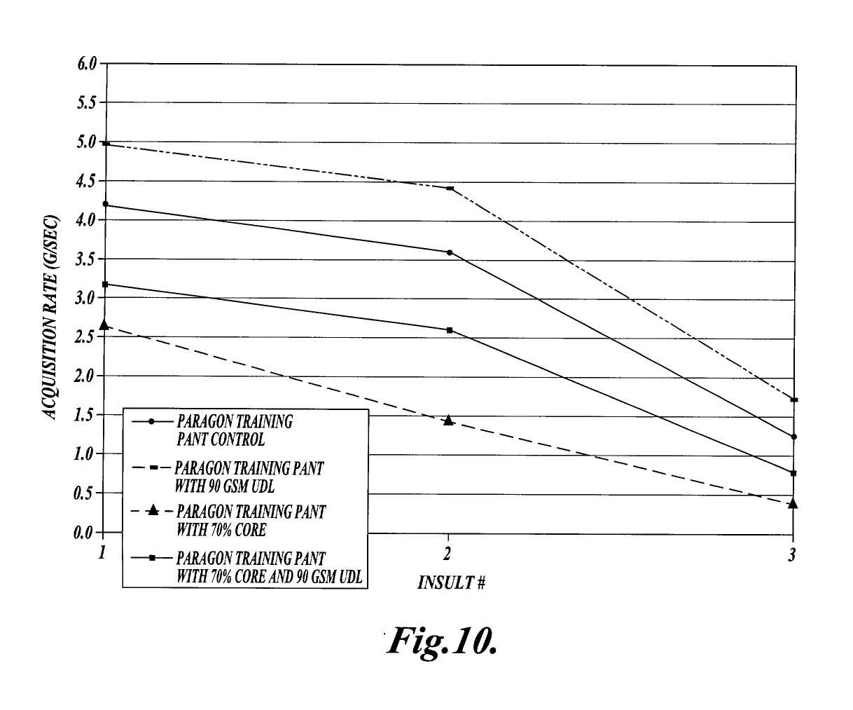

FIG. 9 shows the acquisition rate of the 3^{rd} insult in grams per second. FIG. 10 shows the acquisition rate for three successive insults in grams per second.

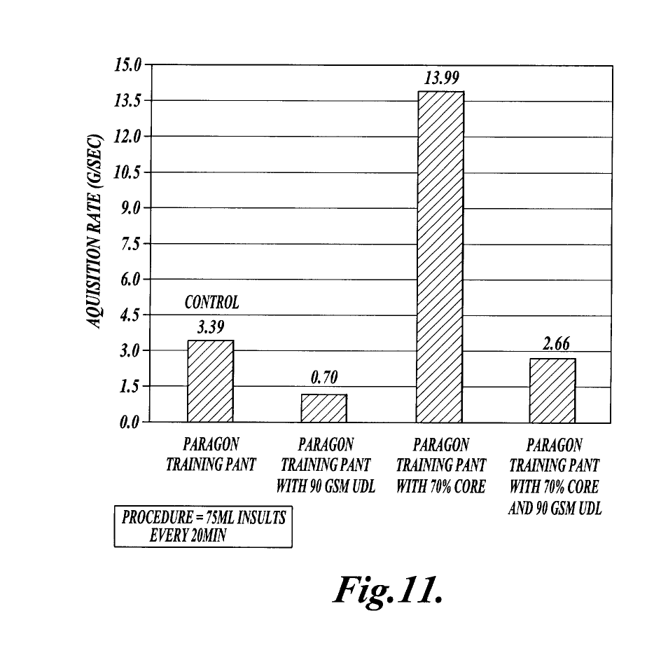

Rewet is reported as the amount of liquid (grams) absorbed back into the filter papers after each liquid dose (i.e., difference between the weight of wet filter papers and the weight of dry filter papers). FIG. 11 shows the rewet after the 4^{th} insult.

### Pulp Extract Surface Tension Method

The following method is used to determine the surface tension of pulp extracts. In the method, pulp fibers are mixed with water to extract residue and contaminants. The surface tension of the filtrate is measured to demonstrate the surface activity of the extractives and their relative concentration on the pulp fibers. The procedure is described below.

A. Wearing gloves to prevent contamination, remove a 2.0 gram subsample of pulp from a pulp sheet and place in a clean, dry 125-mL Nalgene bottle.

B. Add 100 mL of deionized water and cap the bottle tightly.

C. Place the bottle on a wrist action shaker and shake on high intensity for 1 hour.

D. Remove the bottle from the shaker and allow to stand for 10 minutes. This helps to separate the fibers from the water before filtering.

E. Assemble a filtration apparatus using a clean, dry 125-mL Nalgene bottle inside a filter box with an 11.0 cm Buchner funnel placed on top. Place an 11.0 cm Whatman grade #4 filter paper in the Buchner funnel. An equivalent filter can be used if it has the following specifications: fast qualitative type, 12 sec./100 mL filtration speed, 0.06% ash content, and 20-25 μ particle size retention.

F. Attach the filter assembly to a standard (25 in. of Hg) vacuum system.

G. Turn on the vacuum system, uncap the sample bottle, and pour the contents onto the filter in the Buchner funnel. All the filtrate should be removed from the pulp fibers in 15-30 seconds.

H. Turn off the vacuum system and remove the collection bottle from the filter box. Swirl the filtrate in the bottle to ensure thorough mixing.

I. Calibrate the Rosano plate surface tensiometer by using deionized water at room temperature (25° C.) and the platinum plate labeled for surfactants. Condition the plate by dipping in acetone and passing through the flame of a bunsen burner until it glows red. Allow the plate to cool for 10 seconds before using. Conditioning must take place between every sample and every sample replicate.

J. Pour 20 mL of deionized water into a clean, dry 25-mL glass petri dish. Measure the surface tension and perform a duplicate. The surface tension of deionized water at 25° C. is 71.8 dynes/cm. The surface tensiometer is calibrated if each duplicate reading is 71.8±1 dynes/cm.

K. Using the filtrate in the sample bottle, pour 20 mL aliquotes into three clean, dry 25-mL petri dishes.

L. Measure the surface tension of each replicate and report the average. Each replicate should be within±2 dynes/cm. A replicate should be repeated if bubbles are on the surface or within the solution: bubbles adversely affect the reading.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A fibrous layer, comprising a refined blend of crosslinked cellulosic fibers and noncrosslinked cellulosic fibers.

2. The layer of claim 1, wherein the crosslinked cellulosic fibers are present in an amount from about 50 to about 90 percent by weight based on the total weight of the layer.

3. The layer of claim 1, wherein the crosslinked cellulosic fibers are present in an amount from about 75 to about 90 percent by weight based on the total weight of the layer.

4. The layer of claim 1, wherein the crosslinked cellulosic fibers are present in about 85 percent by weight based on the total weight of the layer.

5. The layer of claim 1, wherein the noncrosslinked cellulosic fibers are present in an amount from about 10 to about 50 percent by weight based on the total weight of the layer.

6. The layer of claim 1, wherein the noncrosslinked cellulosic fibers are present in an amount from about 10 to about 25 percent by weight based on the total weight of the layer.

7. The layer of claim 1, wherein the noncrosslinked cellulosic fibers are present in about 15 percent by weight based on the total weight of the layer.

8. The layer of claim 1, wherein the noncrosslinked cellulosic fibers comprise southern pine fibers.

9. The layer of claim 1, wherein the noncrosslinked cellulosic fibers comprise hardwood fibers.

10. The layer of claim 1, wherein the noncrosslinked cellulosic fibers comprise eucalyptus fibers.

11. The layer of claim 4, comprising from about 5 to about 15 percent by weight refined southern pine fibers.

12. The layer of claim 4, comprising up to about 10 percent by weight southern pine fibers.

13. The layer of claim 11, wherein the refined southern pine fibers have a Canadian Standard Freeness of about 500.

14. The layer of claim 4, comprising from about 3 to about 5 percent by weight hardwood fibers.

15. The layer of claim 4, comprising from about 10 to about 12 percent by weight southern pine fibers.

16. The layer of claim 1 further comprising a wet strength agent.

17. The layer of claim 1 having an extracts' surface tension greater than about 50 dynes/cm.

18. The layer of claim 1 having a softness less than about 1200 g.

19. The layer of claim 1 having a mid-point desorption pressure greater than about 120 cm.

20. The layer of claim 1 having a mid-point acquisition pressure less than about 25 cm.

21. The layer of claim 1 having a mid-point uptake greater than about 5 g/g.

22. The layer of claim 1 having a tensile strength greater than about 10 N/50 mm.

23. The layer of claim 1 having a machine direction tear strength greater than about 205 mN.

24. The layer of claim 1 having a cross-machine direction tear strength greater than about 260 mN.

25. An absorbent construct, comprising a liquid distribution layer and a liquid storage layer, wherein the distribution layer comprises a refined blend of crosslinked cellulosic fibers and noncrosslinked cellulosic fibers.

26. The construct of claim 25, wherein the crosslinked fibers are present in an amount from about 50 to about 90 percent by weight based on the total weight of the layer.

27. The construct of claim 25, wherein the crosslinked fibers are present in about 85 percent by weight.

28. The construct of claim 25, wherein the noncrosslinked fibers are present in about 10 to about 50 percent by weight based on the total weight of the layer.

29. The construct of claim 25, wherein the noncrosslinked fibers are present in about 15 percent by weight.

30. The construct of claim 25, wherein the storage layer comprises superabsorbent material.

31. A fibrous layer having a mid-point desorption pressure greater than about 20 cm.

32. The layer of claim 31 having a mid-point desorption pressure greater than about 30 cm.

33. The layer of claim 31 having a mid-point desorption pressure greater than about 40 cm.

34. An absorbent construct, comprising a liquid distribution layer and a liquid storage layer, wherein the distribution layer has a mid-point desorption pressure greater than about 20 cm.

35. The construct of claim 34 having a mid-point desorption pressure greater than about 30 cm.

36. The layer of claim 34 having a mid-point desorption pressure greater than about 40 cm.

37. An absorbent article, comprising any one of the layers of claims 1 or 31.

38. An absorbent article, comprising any one of the constructs of claims 25 or 34.

39. The absorbent article of claim 37, wherein the article is at least one of an infant diaper, a training pant, and an adult incontinence product.

40. The absorbent article of claim 38, wherein the article is at least one of an infant diaper, a training pant, and an adult incontinence product.
